SEQUENCE LISTING (1 sequence)

SEQ ID NO: 1 - DNA, 81 bp, Artificial Sequence
  synthetic
atccagagtgacgcagcatgcttaaggggggggcgggttaagggagtggggagggagctggtgtggacacggtggcttagt

(19) United States
(12) Patent Application Publication
(10) Pub. No.: US 2016/0312265 A1
(43) Pub. Date: Oct. 27, 2016

(54) USE OF NUCLEIC ACID AGENTS FOR ULTRA-SENSITIVE DIGITAL DETECTION AND QUANTIFICATION OF TARGET MOLECULES

(71) Applicant: APTITUDE MEDICAL SYSTEMS, INC., Santa Barbara, CA (US)

(72) Inventors: Jinpeng Wang, Goleta, CA (US); Qiang Gong, Goleta, CA (US); B. Scott Ferguson, Santa Barbara, CA (US)

(73) Assignee: Aptitude Medical Systems, Inc., Santa Barbara, CA (US)

(21) Appl. No.: 15/107,768

(22) PCT Filed: Jan. 14, 2015
(86) PCT No.: PCT/US15/11464

§ 371 Date: Jun. 23, 2016

Related U.S. Application Data
(60) Provisional application No. 61/927,213, filed on Jan. 14, 2014.

(51) Int. Cl. C12Q 1/68; G01N 33/543; G01N 33/53

(52) U.S. Cl. CPC C12Q 1/6804; G01N 33/5308; G01N 33/54386; G01N 2333/525

(57) ABSTRACT

Described herein are methods and systems for detecting and quantifying target molecules and particles in a sample using a plurality of capture objects and a plurality of detection objects. The methods and system utilize nucleic-acid aptamers and digital DNA amplification and detection technology to transform the analog signals obtained from conventional ELISA to digital signals from single molecule counting.

### FIELD OF INVENTION

Described herein are methods and systems for detecting and quantifying analyte molecules in a sample using a plurality of capture objects such as antibodies and a plurality of detection objects such as aptamers.

### BACKGROUND OF THE INVENTION

All publications cited herein are incorporated by reference in their entirety to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Almost all methods for detecting a target molecule are analog in nature, with an average signal measured from the total sample volume. For example, with either absorbance or fluorescence, concentration of the targets being measured is inferred from the absorbance or fluorescence signal, which is a function of the amount of absorbing or fluorescing species present. The current gold standard for measuring specific protein quantity is Enzyme-Linked Immunosorbent Assay (ELISA), which requires large volumes that ultimately dilute reaction product, requiring millions of enzyme labels to generate signals that are detectable utilizing conventional plate readers. Thus, to generate 1 million enzyme labels in a 100 μL volume, the concentration of the target analyte needs to be at least at the sub-picomolar (i.e., pg/mL, assuming 50 kDa molecular weight). Sensitivity of ELISA is therefore limited to the picomolar range and above. It is estimated that only 10% of the proteins in human serum can be detected with currently available approaches. Furthermore, proteins and metabolites that are of most biological interest are present in low abundance. As a result, the discovery of useful protein and metabolite biomarkers and biomarker patterns has been limited.

Single molecule counting is digital in nature, where each target molecule generates a signal that can be counted. It is much easier to measure the presence or absence of a signal than to quantify the absolute amount of signal. Digitizing ELISA signal therefore has the potential of significantly improving the limit of detection of protein analysis. Amplification is an essential part of all single molecule detection techniques. One method of amplification for protein detection is to use an enzyme label that can generate many molecules of detectable product by catalyzing the conversion of substrate molecules into detectable product molecules. Another way to amplify single molecules is by replicating the molecules of interest. This approach is the basis for many nucleic acid detection methods in which a single molecule is amplified using, for example, the polymerase chain reaction (PCR), generating thousands to millions of copies of a particular DNA sequence. The sensitivities achieved by methods for detecting nucleic acid beat those for proteins by orders of magnitude since the amplification of nucleic acids is much more efficient and robust.

Described herein are methods and systems that utilize digital nucleic acid detection and quantification methods for protein analysis. Specifically, nucleic-acid aptamer and digital DNA amplification and detection technologies are used to transform the analog signals obtained from conventional ELISA to digital signals for single molecule counting. The methods and systems described herein utilize aptamers as translators between the protein domain and the nucleic acid domain. Aptamers are synthetic nucleic acid based affinity reagents that can bind to their target molecules with high affinity and specificity. Herein, aptamers are used instead of antibodies as the detection reagent in a sandwich immunoassay. The aptamers not only serve as the detection affinity reagent, but also as a “translator” from proteins to nucleic acids. Because the nucleic acid aptamer can be easily amplified through an enzymatic reaction, digital nucleic acid amplification and detection technology may be used for protein detection, which can significantly improve the assay sensitivity. The methods described herein improve over ELISA because both the translation from protein domain to nucleic acid domain and the digitization of the measurement yields unprecedented assay performance.

### SUMMARY OF THE INVENTION

The following embodiments and aspects thereof are described and illustrated in conjunction with systems, compositions and methods which are meant to be exemplary and illustrative, not limiting in scope.

Provided herein are methods for detecting target molecules or particles in a sample. The methods include exposing the sample to a plurality of capture objects that each include a binding surface having affinity for the target molecules or particles, so as to form a complex between the capture objects and the target molecules or particles; removing the capture objects not complexed with the target molecules or particles; exposing the complex of capture object and target molecules or particles to a plurality of detection objects so as to form a complex between the capture objects, target molecules or particles and the detection objects; removing the detection objects not complexed with the capture objects and the target molecules or particles; eluting the detection objects complexed with the capture objects and the target molecules or particles; partitioning the detection objects into compartments; and detecting the presence or absence of the detection objects in each compartment, so as to detect the target molecules or particles in the sample.

Also described herein are methods for detecting target molecule in a sample. The methods include exposing the sample to a solid support comprising a capture antibody specific for the target molecule so as to form a target-antibody complex; removing the unbound sample and antibody; exposing the target-antibody complex to a detection aptamer specific for the target; removing the unbound aptamer; eluting the aptamer bound to the target-antibody complex; partitioning the aptamers into compartments; and detecting the presence or absence of an aptamer in each compartment, so as to detect the target molecules in the sample.

Further provided is a system for detecting target molecules or particles in a sample. The system includes an array of reaction vessels wherein at least one of the reaction vessels contain no sample and at least one of the vessels contains a control sample that does not contain a target molecule or particle; a plurality of capture objects that each include a binding surface having affinity for the target molecules or particles, so as to form a complex between the capture objects and the target molecules or particles; plurality of detection objects so as to form a complex between the capture objects, target molecules or particles and the detection objects; and an agent to elute the detection objects complexed with the capture objects and the target molecules or particles.

In various embodiments, the sample is a fluid sample and may include but is not limited to blood, plasma or urine.

In some embodiments, the target is a protein or a nucleic acid or a combination thereof. The protein may be a monomer or a multimer. Additional targets may include but are not limited to, for example, small molecules, amino acids, carbohydrates, lipids, aminoglycosides, antibiotics, peptides, proteins, post-translational modification, nucleic acids or combinations thereof.

In some embodiments, the plurality of capture objects is any one or more of an antibody, an aptamer, a polypeptide, receptor, ligand, small molecule, or any other affinity reagents for the target molecules or a combination thereof. In some embodiments, the aptamer is a nucleic acid (DNA, RNA, XNA (nucleic acid analogs)) aptamer. In some embodiments, the aptamer is a peptide aptamer.

In some embodiments, the plurality of detection objects includes but is not limited to any one or more of an antibody, an aptamer, a polypeptide, receptor, ligand, small molecule, or any other affinity reagents for the target molecules or a combination thereof. In some embodiments, the aptamer is a nucleic acid (DNA, RNA, XNA (nucleic acid analogs)) aptamer. In some embodiments, the aptamer is a peptide aptamer.

In some embodiments, the plurality of detection objects includes DNA conjugated to affinity reagents. In exemplary embodiments, DNA may be conjugated to any one or more of antibody, polypeptide, receptor, ligand, small molecule or any other affinity reagents appropriate for the target molecule. In various embodiments, the DNA serves as the signal molecule for the digital detection step.

In some embodiments, the detection object is one or more aptamers. The aptamer may be conjugated to an affinity reagent. In exemplary embodiments, the aptamer may be conjugated to any one or more of antibody, polypeptide, receptor, ligand, small molecule or any other affinity reagents appropriate for the target molecule. In some embodiments, the aptamer is a nucleic acid (DNA, RNA, XNA (nucleic acid analogs)) aptamer. In some embodiments, the aptamer is a peptide aptamer. In various embodiments, the aptamer serves as the signal molecule for the digital detection step.

In some embodiments, the plurality of capture objects are antibodies and the plurality of detection objects are aptamers. In some embodiments, the plurality of capture objects are aptamers and the plurality of detection objects are aptamers. In some embodiments, the aptamer may be conjugated to an affinity reagent. In some embodiments, the aptamer is not conjugated to an affinity reagent. In various embodiments, the aptamer serves as the signal molecule for the digital detection step.

The plurality of capture objects may be bound to a plurality of solid support. In various aspects, the plurality of solid support includes but is not limited to beads, nanoparticles, nanotubes (e.g., carbon nanotubes), microtiter plates, microfluidic channels, electrodes, vesicles, cells, film (e.g. nitrocellulose), tubing, or combinations thereof.

In some embodiments, aptamer is detected by digital detection methods. The digital detection methods are any one or more of digital polymerase chain reaction (PCR), digital rolling circle amplification (RCA), digital loop-mediated amplification (LAMP), Recombinase Polymerase Amplification (RPA) or digital nucleic acid sequence based amplification (NASBA).

In some aspects, the eluted detection objects are partitioned into compartments such that each compartment consists of zero or one detection object. The compartment with one detection object is indicative of the presence of one target molecule. In some aspects, an absolute concentration of the target molecule in the sample is the total number compartments with one aptamer divided by the volume of the sample.

In some embodiments, the concentration of target molecules or particles in the sample is less than about 50×10⁻¹⁵M, or less than about 40×10⁻¹⁵M, or less than about 30×10⁻¹⁵M, or less than about 20×10⁻¹⁵M, or less than about 10×10⁻¹⁵M, or less than about 5×10⁻¹⁵M, or less than about 1×10⁻¹⁵M. The concentration of target molecules or particles in the fluid sample may be determined at least in part by comparison of a measure parameter to a calibration standard.

### BRIEF DESCRIPTION OF FIGURES

Exemplary embodiments are illustrated in the referenced figures. It is intended that the embodiments and figures disclosed herein are to be considered illustrative rather than restrictive.

FIG. 1 depicts, in accordance with various embodiments of the present invention, a schematic of an aptamer-based digital detection and quantification. (a) Target molecules are captured on beads from an initial sample and labeled using detection aptamers. Detection aptamers correspond to a one-to-one representation of the target analyte, and were then eluted from the bead surface. (b) Single DNA molecule counting is performed by dividing the eluted detection aptamers into hundreds to even millions of separate compartments (e.g., water-in-oil droplets) such that each compartment will contain either 0 or only 1 detection aptamer. Endpoint amplification is performed and the number of fluorescent reactions counted. Amplification-positive, “bright” reactions each contained 1 target molecule, and amplification-negative, “dark” reactions have no target

### DETAILED DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3rd ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

As used herein, “support” or “solid support” refers to materials which the capture objects are bound to, either directly or indirectly, prior to exposing the capture objects to the sample.

As used herein, “capture objects” refer to objects that bind to the target molecules or particles in the sample. The target molecules or particles are the analytes of interest which are to be detected and/or quantitated. The capture objects are used to isolate the target molecules or particles of interest from the remaining sample. When bound, the capture objects form a complex with the target molecules or particles.

As used herein, “detection objects” refers to objects that bind to the target molecules or particles at different locations on the target molecules or particles compared to the binding locations of the capture objects on the target molecules or particles. The detection objects may bind to target molecule or particles when complexed or when not complexed with the capture objects. The detection objects are detected and quantified and the amount of the detection objects is indicative of the amount of the target molecules or particles in the original sample.

The ability to detect and quantify non-nucleic acid molecules (protein and small molecule analytes) has lagged behind the ability to analyze nucleic acids. This is because of a unique property of nucleic acids: they can be exponentially amplified by enzymes to generate thousands to millions of copies using, for example, the polymerase chain reaction (PCR). In addition, because the amplification of nucleic acids is efficient and robust, the measurement of nucleic acids can be digitized using single molecule counting, where each DNA molecule is isolated into a discrete spatial unit and amplified to generate a signal countable unit. This digitization enables the detection of single molecules, hence unprecedented quantification and sensitivity. However, this capability has not been extended to non-nucleic acid targets because enzymatic exponential amplification is not possible for proteins and small molecules. The methods and systems described herein overcome this limitation and enable a universal digital detection and quantification method for any target molecule. The method exploits nucleic-acid aptamers, as well as and digital DNA amplification and detection technology to transform the analog signals obtained from conventional ELISA to digital signals from single molecule counting.

There have been previous attempts to use nucleic acid quantification methods for protein analysis. However, these have focused on the replication of nucleic acid labels on proteins. For example, a protein target amplification technique, called immuno-PCR, makes use of oligonucleotide labels, which can subsequently be detected using quantitative PCR. In immuno-PCR, the DNA label is copied until it produces a certain level of signal; the number of amplification cycles needed to reach this point is then used to calculate how many DNA labels were originally present in the sample. While the immuno-PCR method improves the sensitivity of protein detection, it cannot distinguish copy number variants smaller than twofold and can be prone to false-positive signal generation. Furthermore, the coupling between antibody and DNA requires an extremely careful optimization process for each new target and the number of DNA labels per antibody is not controllable and inconsistent from batch to batch.

In contrast, the methods described herein use nucleic-acid aptamers as the detection affinity reagent to serve as a translator from protein detection to nucleic acid single molecule counting. The digital single molecule counting technology transforms the exponential, analog signals obtained from conventional qPCR to linear, digital signals, which can significantly improve the assay sensitivity and resolution for quantification. The precise number of copies of detection aptamers in the original sample can be ascertained because as described herein, a precise ratio of one aptamer to one protein is employed, the copy number of the target molecule (for example, protein) in the original sample is determined. The absolute concentration of the target is easily calculated as being equal to the total number of target molecules divided by the total measured volume.

Rissin et al. (Nat Biotechnol. 2010 June; 28(6): 595-9 Single-molecule enzyme-linked immunosorbent assay detects serum proteins at subfemtomolar concentrations) recently developed a platform (termed digital ELISA) to digitize ELISA signal for quantification of protein concentration. Their approach uses large numbers of beads (200,000-500,000 beads) to ensure both that a high proportion (approximately 70%) of analyte molecules in a sample are captured and that most beads capture only a single analyte molecule. The captured analyte molecules are subsequently bound by a second, enzyme-labeled antibody to form an antigen/antibody sandwich. Beads (approximately 5%-15% of the beads) are physically isolated and the association between the beads and the labeled protein is determined, so as to digitize the ELISA signal. Digitization of the sandwich complex is achieved by loading the beads into arrays of femtoliter wells designed to hold a single bead in the presence of substrate, then physically sealing the wells off (using either a rubber gasket or oil) and allowing the reaction of single enzyme molecules with their substrate to develop (typically for 30 s to 2.5 min). Image analysis is then used to identify those wells that contain beads and the fluorescence intensity of those wells at a specific wavelength.

In contrast to digital ELISA, the methods and systems described herein use aptamers as both the detection affinity reagent and a translator from protein concentration measurement to single molecule counting of nucleic acid aptamers. Since nucleic acids can be exponentially amplified by enzymes to generate thousands to millions of copies, various digital DNA amplification and detection techniques can be used here, including but not limited to digital polymerase chain reaction (PCR), digital rolling circle amplification (RCA), digital loop-mediated amplification (LAMP), or digital nucleic acid sequence based amplification (NASBA). As detailed below, after an antibody-analyte (target)-aptamer sandwich complex is formed on the solid support, the detection aptamers are eluted and partitioned into hundreds or even millions of separate reactions so that each compartment contains only one or no copies of the detection aptamer. After “dividing”, one of the above amplification methods is performed to endpoint. By counting the number of “positive” (or “1”) compartments (in which the detection aptamer is amplified and detected) versus “negative” (or “0”) compartments (in which it is not), the inventors can determine exactly how many copies of the detection aptamer were in the original sample, and due to one aptamer per protein relationship, we then also know how many copies of the target protein analyte were in the initial sample as well. Thus, the absolute concentration of the target is easily calculated as being equal to the total number of target molecules divided by the total measured volume.

Systems and methods for the detection and/or quantification of target molecules (such as proteins and nucleic acids), particles (such as, for example, cells, cellular organelles and other biological or non-biological particulates) and the like, in a sample are described herein.

Specifically, provided herein is a method for detecting target molecules or particles in a sample. The method comprises exposing the sample to a plurality of capture objects that each include a binding surface having affinity for the target molecules or particles, so as to form a complex between the capture objects and the target molecules or particles; removing the capture objects not complexed with the target molecules or particles; exposing the complex of capture object and target molecules or particles to a plurality of detection objects so as to form a complex between the capture objects, target molecules or particles and the detection objects; removing the detection objects not complexed with the capture objects and the target molecules or particles; eluting the detection objects complexed with the capture objects and the target molecules or particles; partitioning the detection objects into compartments; and detecting the presence or absence of the detection objects in each compartment, so as to detect the target molecules or particles in the sample.

Also provided herein is a method for detecting target molecule in a sample. The method comprises exposing the sample to a solid support comprising a capture antibody specific for the target molecule so as to form a target-antibody complex; removing the unbound sample and antibody; exposing the target-antibody complex to a detection aptamer specific for the target; removing the unbound aptamer; eluting the aptamer bound to the target-antibody complex; partitioning the aptamers into compartments; and detecting the presence or absence of an aptamer in each compartment, so as to detect the target molecules in the sample.

Further provided are systems and kits for detecting target molecules or particles in a sample. The systems and kits comprise an array of reaction vessels wherein at least one of the reaction vessels contain no sample and at least one of the vessels contains a control sample that does not contain a target molecule or particle; a plurality of capture objects that each include a binding surface having affinity for the target molecules or particles, so as to form a complex between the capture objects and the target molecules or particles; plurality of detection objects so as to form a complex between the capture objects, target molecules or particles and the detection objects; and an agent to elute the detection objects complexed with the capture objects and the target molecules or particles.

In various embodiments, the sample is a fluid sample. The sample may be any one or more of buffer, blood, plasma, serum, urine, saliva, and sweat, tears, breast bile, bile, cerebral spinal fluid, sputum, lavage, lymph, cell lysate, liquefied tissue, swab eluent, liquefied plant matter, food products, waste water, wash water, drinking water. The sample is exposed to the capture objects and the detection objects in a reaction vessel.

The reaction vessel may be any one or more of a test tube, centrifuge tube, column, a microarray plate, a microtiter plate, microfluidic devices, tubing, tubes coated with the capture objects. In some embodiments, the vessels are coated with capture objects. In some embodiments, the vessels are not coated with capture objects.

The sample volume may be any of about 1 μl to about 20 μl, about 20 μl to 40 μl, about 40 μl to about 60 μl, about 60 μl to about 80 μl, about 80 μl to about 100 μl, about 100 μl to about 500 μl, about 500 μl to about 1 μl, about 1 μl to about 5 μl, about 5 μl to about 10 μl, about 10 μl to about 50 μl, about 50 μl to about 100 μl or a combination thereof. The original sample may be undiluted to diluted for use in the methods described herein. The sample may be diluted 1-fold, 2-fold, 5-fold, 10-fold, 50-fold, 100-fold, 100-fold or a combination thereof.

In various embodiments, the concentration of target molecules or particles in the sample is less than about 50×10⁻¹⁵M, or less than about 40×10⁻¹⁵M, or less than about 30×10⁻¹⁵M, or less than about 20×10⁻¹⁵M, or less than about 10×10⁻¹⁵M, or less than about 5×10⁻¹⁵M, or less than about 1×10⁻¹⁵M. In some embodiments, the concentration of target molecules or particles in the sample is in the attomole range.

### Target Molecules or Particles

As will be appreciated by those in the art, a large number of target molecules and particles may be detected and quantified using methods and systems of the present invention. Any target molecule that is able to be made to become immobilized with respect to a capture object (e.g., via a binding surface comprising a plurality of capture components) can be potentially investigated using the methods and systems described herein. Certain specific targets of potential interest that may comprise a target molecule are described below. The list below is exemplary and non-limiting.

In some embodiments, the target molecule or particle are one or more of proteins, nucleic acids, peptides, carbohydrates, small molecules, viral bacteria, bacteria or a combination thereof. In various embodiments, the target protein may be a monomer or a multimer. It should be understood that while much of the discussion below is directed to target molecules that are proteins, this is by way of example only and other materials (such as target particles) may be detected and/or quantified.

In some embodiments, the target molecule or particle (for example, a target protein) may be associated with various diseases including but not limited to cancer, infectious disease, inflammatory diseases, neuronal disorders, diabetes, cardiovascular diseases, hematologic diseases, autoimmune diseases, inflammatory diseases or combination thereof.

Examples of target molecules or particles associated with cancer include but are not limited to any one or more of 4-1BB, 5T4, adenocarcinoma antigen, alpha-fetoprotein, BAFF, B-lymphoma cell, C242 antigen, CA-125, carbonic anhydrase 9 (CA-IX), C-MET, CCR4, CD152, CD19, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNTO888, CTLA-4, DR5, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgG1, L1-CAM, IL-13, IL-6, insulin-like growth factor I receptor, integrin α5β1, integrin αvβ3, MORAb-009, MS4A1, MUC1, mucin CanAg, N-glycolylneuraminic acid, NPC-1C, PDGF-Rα, PDL192, phosphatidylserine, prostatic carcinoma cells, RANKL, RON, ROR1, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, vimentin or combinations thereof. Other target molecules or particles specific for cancer will be apparent to those of skill in the art and may be used in connection with alternate embodiments of the invention

Examples of target molecules or particles associated with inflammatory diseases include but are not limited to any one or more of AOC3 (VAP-1), CAM-3001, CCL11 (eotaxin-1), CD125, CD147 (basigin), CD154 (CD40L), CD2, CD20, CD23 (IgE receptor), CD25 (α chain of IL-2 receptor), CD3, CD4, CD5, IFN-α, IFN-γ, IgE, IgE Fc region, IL-1, IL-12, IL-23, IL-13, IL-17, IL-17A, IL-22, IL-4, IL-5, IL-5, IL-6, IL-6 receptor, integrin α4, integrin α4β7, Lama glama, LFA-1 (CD11a), MEDI-528, myostatin, OX-40, rhuMAb β7, scleroscin, SOST, TGF beta 1, TNF-α or VEGF-A. Other target molecules or particles specific for inflammatory diseases will be apparent to those of skill in the art and may be used in connection with alternate embodiments of the invention.

Examples of target molecules or particles associated with neuronal disorders include but are not limited to any one or more of beta amyloid or MABT5102A. Other target molecules or particles specific for neuronal disorders will be apparent to those of skill in the art and may be used in connection with alternate embodiments of the invention.

Examples of target molecules or particles associated with diabetes include but are not limited to any one or more of L-1β or CD3. Other target molecules or particles specific for diabetes or other metabolic disorders will be apparent to those of skill in the art and may be used in connection with alternate embodiments of the invention.

Examples of target molecules or particles associated with cancer include but are not limited to any one or more of C5, cardiac myosin, CD41 (integrin alpha-IIb), fibrin II, beta chain, ITGB2 (CD18) and sphingosine-1-phosphate. Other antigens specific for cardiovascular diseases will be apparent to those of skill in the art and may be used in connection with alternate embodiments of the invention.

Examples of target molecules or particles associated with infectious diseases include but are not limited to any one or more of anthrax toxin, CCR5, CD4, clumping factor A, cytomegalovirus, cytomegalovirus glycoprotein B, endotoxin, Escherichia coli, hepatitis B surface antigen, hepatitis B virus, HIV-1, Hsp90, Influenza A hemagglutinin, lipoteichoic acid, Pseudomonas aeruginosa, rabies virus glycoprotein, respiratory syncytial virus and TNF-α. Other target molecules or particles specific for infectious diseases will be apparent to those of skill in the art and may be used in connection with alternate embodiments of the invention.

In various embodiments, at least about 10%, or at least about 20%, or at least about 30%, or at least about 40%, or at least about 50%, or at least about 60%, or at least about 70%, or at least about 80%, or at least about 90%, or at least about 95%, or at least about 99% or at least about 100% of the target molecules or particles bind to the capture objects so as to form a complex between the capture objects and the target molecules or particles.

### Support

The plurality of capture objects are bound to a support either directly or indirectly (for example, via a linker). In some embodiments, the linker may comprise any moiety, or modification of the binding surface of the support that facilitates the attachment of the capture object to the surface. The linkage between the capture object and the surface of the support may comprise one or more chemical or physical (e.g., non-specific attachment via van der Waals forces, hydrogen bonding, electrostatic interactions, hydrophobic/hydrophilic interactions; etc.) bonds and/or chemical linkers providing such bond(s).

In certain embodiments, the surface of the support may also comprise a protective or passivating layer that can reduce or minimize non-specific attachment of non-capture components (e.g., target molecules or particles, binding ligands) to the binding surface during the assay which may lead to false positive signals during detection or to loss of signal. In certain embodiments, examples of materials that may be utilized to form passivating layers include, but are not limited to: polymers, such as poly(ethylene glycol), that repel the non-specific binding of proteins; naturally occurring proteins with such a property, such as serum albumin and casein; surfactants, e.g., zwitterionic surfactants, such as sulfobetaines; naturally occurring long-chain lipids; and nucleic acids, such as salmon sperm DNA.

Examples of solid support include but are not limited to a plurality of beads, nanotubes (e.g., carbon nanotubes), microtiter plates, microfluidic channels, electrodes, vesicles, cells, film (e.g. nitrocellulose), tubing, or combinations thereof. In certain embodiments, the beads have an average diameter between about 0.1 micrometer and 100 micrometers, between about 1.0 micrometer and 10 micrometers, 0.01 micrometer and 1 micrometer. The ratio of beads to capture objects may depend on the size of the beads. For example, a 1 μM bead may have between 10⁴ and 10⁶ capture objects. The plurality of beads may have a variety of properties and parameters. For examples, the beads may be magnetic, polystyrene, fluorescent, agarose, sepharose, silicon, silicon oxide, or a combination thereof. In an exemplary embodiment, beads are coated with capture antibodies specific to the target protein.

In various embodiments, the solid support can be of any shape, e.g., sphere-like, disks, rings, cube-like, etc.), a dispersion or suspension of particulates (e.g., a plurality of particles in suspension in a fluid), nanotubes, or the like. In some embodiments, the support is insoluble or substantially insoluble in the solvent(s) or solution(s) utilized in the assay. In some cases, the support is solid or substantially solid (e.g., is essentially free of pores), however, in some cases, the support may be porous or substantially porous, hollow, partially hollow, etc. The plurality of support substances (such as solid support) may be non-absorbent, substantially non-absorbent, substantially absorbent, or absorbent. In some cases, the solid support may comprise a magnetic material, which as described herein, may facilitate certain aspect of the assay (e.g., washing step). In some cases, the support surface may also comprise a protective or passivating layer that can reduce or minimize non-specific binding events (e.g., analyte molecules, binding ligands, etc.).

In various embodiments, the method of attachment of the capture objects to the surface of the support (for example, a solid support) depends of the type of linkage employed and may potentially be accomplished by a wide variety of suitable coupling chemistries/techniques known to those of ordinary skill in the art. The particular means of attachment selected will depend on the material characteristics of the surface of the support and the nature of the capture object. In certain embodiments, the capture objects may be attached to the support surface through the use of reactive functional groups on each. According to one embodiment, the binding surface may be derivatized such that a chemical functionality is presented at the binding surface on the support which can react with a chemical functionality on the capture objects resulting in attachment. Examples of functional groups for attachment that may be useful include, but are not limited to, amino groups, carboxy groups, epoxide groups, maleimide groups, oxo groups, and thiol groups. Functional groups can be attached, either directly or through the use of a linker, the combination of which is sometimes referred to herein as a “crosslinker.” Crosslinkers are known in the art; for example, homo- or hetero-bifunctional crosslinkers as are well known (e.g., see 1994 Pierce Chemical Company catalog, technical section on crosslinkers, pages 155-200, or “Bioconjugate Techniques” by Greg T. Hermanson, Academic Press, 1996). Non-limiting example of crosslinkers include alkyl groups (including substituted alkyl groups and alkyl groups containing heteroatom moieties), esters, amide, amine, epoxy groups and ethylene glycol and derivatives. A crosslinker may also comprise a sulfone group, forming a sulfonamide.

In some embodiments, the functional group is a light-activated functional group. For example, the functional group can be activated by light to attach the capture object to the surface of the support (for example, a solid support). One example is PhotoLink™ technology available from SurModics, Inc. in Eden Prairie, Min.

In some embodiments, the support may comprise streptavidin-coated surfaces and the capture objects may be biotinylated. Exposure of the capture object to the streptavidin-coated support surfaces can cause association of the capture object with the support surface by interaction between the biotin component and streptavidin.

In some embodiments, attachment of the capture objects to the surface of the support may be effected without covalently modifying the binding surface of a capture object. For example, the attachment functionality can be added to the binding surface by using a linker that has both a functional group reactive with the capture component and a group that has binding affinity for the binding surface. In certain embodiments, a linker comprises a protein capable of binding or sticking to the binding surface; for example, in one such embodiment, the linker is serum albumin with free amine groups on its surface. A second linker (crosslinker) can then be added to attach the amine groups of the albumin to the capture object (e.g., to carboxy groups).

### Capture Objects

In various embodiments, the support (for example, solid support) is coated with a plurality of capture objects. The capture objects comprise a surface that attaches to the support (for example, solid support) and a surface that attaches to the target molecule or particle. In various embodiments, plurality of capture objects bind the target molecule or particles. In various embodiments, as is apparent to a person of skill in the art, the type of capture objects used will depend on the nature of the target molecules or particles.

Capture objects for a wide variety of target molecules are known or can be readily found or developed using known techniques. For example, when the target molecule is a protein, the capture objects may comprise proteins, particularly antibodies or fragments thereof (e.g., antigen-binding fragments (Fabs), Fab′ fragments, pepsin fragments, F(ab′)₂ fragments, full-length polyclonal or monoclonal antibodies, antibody-like fragments, etc.), other proteins, such as receptor proteins, Protein A, Protein C, etc., nucleic acids (for example, aptamers) or small molecules. In certain embodiments, capture objects for proteins comprise peptides. For example, when the target molecule is an enzyme, suitable capture objects may include enzyme substrates and/or enzyme inhibitors. In some cases, when the target molecule is phosphorylated or methylated, the capture objects may include a phosphate-binding agent or a methyl-binding agent, respectively. For example, the phosphate-binding agent may include metal-ion affinity media such as those describe in U.S. Pat. Nos. 7,070,921 and 7,632,651. Additionally, when the target molecule is a single-stranded nucleic acid, the capture object may be a complementary nucleic acid. Similarly, the target molecule may be a nucleic acid binding protein and the capture object may be a single-stranded or double-stranded nucleic acid; alternatively, the capture object may be a nucleic acid-binding protein when the target molecule is a single or double stranded nucleic acid. Alternatively, as is generally described in U.S. Pat. Nos. 5,270,163, 5,475,096, 5,567,588, 5,595,877, 5,637,459, 5,683,867, 5,705,337, and related patents, nucleic acid “aptamers” may be developed for capturing virtually any target molecule. Also, for example, when the target molecule is a carbohydrate, potentially suitable capture objects include, for example, antibodies, lectins, and selectins. As will be appreciated by those of ordinary skill in the art, any molecule that can specifically associate with a target molecule of interest may potentially be used as a capture objects.

In some embodiments, suitable target molecule/capture object pairs can include, but are not limited to, antibodies/antigens, receptors/ligands, proteins/nucleic acid, nucleic acids/nucleic acids, enzymes/substrates and/or inhibitors, carbohydrates (including glycoproteins and glycolipids)/lectins and/or selectins, proteins/proteins, proteins/small molecules; small molecules/small molecules, etc. According to one embodiment, the capture objects are portions (particularly the extracellular portions) of cell surface receptors that are known to multimerize, such as the growth hormone receptor, glucose transporters (particularly GLUT 4 receptor), and T-cell receptors and the target analyte molecules are one or more receptor target ligands.

In some embodiments, the capture objects may be any one or more of antibodies specific for the target molecules or particles, aptamers specific to the target molecules or particles, polypeptides specific to the target molecules or particles, receptors, ligands, small molecules, or combinations thereof. In an embodiment, the capture objects are antibodies that specifically bind the target molecule and do not bind non-target molecules.

In some embodiments, the target particle is a biological cell (e.g., mammalian, avian, reptilian, other vertebrate, insect, yeast, bacterial, cell, etc.), and the capture object may be a ligand having specific affinity for a cell surface antigen (e.g., a cell surface receptor). In one embodiment, the capture object is an adhesion molecule receptor or portion thereof, which has binding affinity for a cell adhesion molecule expressed on the surface of a target cell type.

In some embodiments, the binding affinity of the target molecule to its capture object may be between at least about 10⁴ and about 10⁶ M⁻¹, at least about 10⁵ and about 10⁹ M⁻¹, at least about 10⁷ and about 10⁹ M⁻¹, greater than about 10⁹ M⁻¹, or the like.

Those of ordinary skill in the art will be aware of methods and techniques for exposing a plurality of capture objects to a fluid sample containing or suspected of containing a target molecule or particle for initial target capture. For example, the plurality of capture objects may be added (e.g., as a solid, as a solution) directly to a fluid sample. As another example, the fluid sample may be added to the plurality of capture objects (e.g., in solution, as a solid). In some instances, the solutions may be agitated (e.g., stirred, shaken, etc.). The complex formed between the capture objects and the target molecules or particles is separated from the rest of the sample.

In some embodiments, the sample may be washed after exposure and/or incubation with capture objects in a reaction vessel. In this instance, the wash step may be used to wash away any molecules that are not bound to the capture objects. The wash step may be performed by any method known to those skilled in the art, for example, by placing the reaction vessels in a wash solution or by adding wash solution to the reaction vessels. The number of times that the target molecule-capture object complex may be washed will be apparent to a person of skill in the art. In some embodiments, the wash solution may be a solution that does not cause change to the surface of the reaction vessels or the interactions between at least two components of the assay (e.g., a capture object and a target molecule or particle).

### Detection Objects

The detection objects bind to the complexes formed between the target molecules or particles and the capture objects so as to form complexes comprising the target molecules or particles, the capture objects and the detection objects. As will be apparent to a person of skill in the art, the nature of the detection object will depend on the nature of the target molecule and the nature of the capture object.

In some embodiments, the plurality of detection objects includes but is not limited to any one or more of an antibody, an aptamer, a polypeptide, receptor, ligand, small molecule, or any other affinity reagents for the target molecules or a combination thereof. In some embodiments, the aptamer is a nucleic acid (DNA, RNA, XNA (nucleic acid analogs)) aptamer. In some embodiments, the aptamer is a peptide aptamer. Detection objects which are not nucleic-acid based are conjugated with a nucleic acid label. In various embodiments, the aptamers and/or nucleic acids serve as the signal molecule for the digital detection step.

In some embodiments, the plurality of detection objects includes DNA conjugated to affinity reagents. In exemplary embodiments, DNA may be conjugated to any one or more of antibody, polypeptide, receptor, ligand, small molecule or any other affinity reagents appropriate for the target molecule.

In some embodiments, the detection object is one or more aptamers. The aptamer may be conjugated to an affinity reagent. In exemplary embodiments, the aptamer may be conjugated to any one or more of antibody, polypeptide, receptor, ligand, small molecule or any other affinity reagents appropriate for the target molecule. In some embodiments, the aptamer is a nucleic acid (DNA, RNA, XNA (nucleic acid analogs)) aptamer. In an exemplary embodiment, a TNFα specific aptamer consists of the sequence 5′-ATCCAGAGTGACGCAGCATGCTTAAGGGGGGGGCGGGTTAAGGGAGTGGGGAG GGAGCTGGTGTGGACACGGTGGCTTAGT-3′. In an exemplary embodiment, a TNFα specific aptamer comprises the sequence 5′-ATCCAGAGTGACGCAGCATGCTTAAGGGGGGGGCGGGTTAAGGGAGTGGGGAG GGAGCTGGTGTGGACACGGTGGCTTAGT-3′.

In one embodiment, the capture object is an antibody that specifically binds the target protein and the detection object is an aptamer that specifically binds the target protein. The target protein may be a monomer or a multimer. In an exemplary embodiment, if the target protein is a monomer, the antibody and the aptamer bind to two different epitopes on the target protein. In an exemplary embodiment, if the target protein is a multimer, the antibody and the aptamer bind to the same epitope on the target protein.

In another embodiment, the capture object is an aptamer that specifically binds that target protein and the detection object is an aptamer that specifically binds the target protein. The target protein may be a monomer or a multimer. In an exemplary embodiment, if the target protein is a monomer, the capture aptamer and the detection aptamer bind to two different epitopes on the target protein. In an exemplary embodiment, if the target protein is a multimer, the capture aptamer and the detection aptamer bind to the same epitope on the target protein.

In various embodiments, at least about 10%, or at least about 20%, or at least about 30%, or at least about 40%, or at least about 50%, or at least about 60%, or at least about 70%, or at least about 80%, or at least about 90%, or at least about 95%, or at least about 99% or at least about 100% of the complex between the capture objects and the target molecules or particles bind to the detection objects so as to form a complex between the capture objects, target molecules or particles and the detection objects.

### Compartmentalization and Quantification

In various embodiments, the detection objects are eluted away from the complex formed by the target molecules or particles, the capture objects and the detection objects. The eluted detection objects are detected and quantitated and the amount of the detection objects is indicative of the target molecules or particles in original sample. In various embodiments, the eluted detection objects are partitioned across a plurality of reaction sites. The reaction sites may be any of water-in-oil emulsions, double emulsion, microtiter plate, cell (e.g. bacteria), micro-droplets in a microfluidic device, micro- or nano-chambers in a microfluidic device.

In some embodiments, when the detection objects are aptamers, the detection aptamers are eluted and compartmentalized such that there is zero or one aptamer per compartment.

Once eluted, the eluent containing aptamer is diluted and then partitioned into smaller volumes. For example, 100 μL eluent volume could be diluted into 1 mL, and then discretized into 10⁹ compartments that are each 1 pL in volume (droplets of radius of ˜6 μm).

The system described herein enables digital quantification by counting the number of compartments which contain the aptamer. In order for this method to be accurate, there must be a low probability of more than one aptamer existing in a single compartment. To reduce this likelihood the number of compartments must be greater than the number of aptamers in the eluent. Thus, total the number of compartments sets the upper limit of accurate quantification, and dynamic range.

In the above example, if one aptamer is present, it will be easily counted. If there are 100 aptamers, it is highly unlikely that among the 10 billion compartments, one will contain more than one aptamer, so again, these will be easily counted accurately. However, as the number of aptamers approaches the number of droplets, the accuracy decreases. For example at 10 billion aptamers, most compartments will contain more than one aptamer, and thus counting will give an under-representation of the actual quantity. The limit of number of compartments is tied to the total volume which can be processed, and the number of compartments which can be counted. The lower limit of compartment size is set by the ability to accurately sense a positive signal in a small compartment (smaller compartment=lower absolute signal).

The upper limit can be easily expanded by performing serial dilutions of the initial sample volume. The number of compartments can also be decreased by partitioning into volumes of increasing size, wherein signal from each volume, corresponds to likelihood at a specific concentration.

The compartmentalized detection objects may be detected and quantified using any detection methods such as digital PCR, SYBR® family of dyes, assaying changes in turbidity, molecular beacon probes, droplet digital PCR, quantitative PCR, rolling circle amplification (RCA), Recombinase Polymerase Amplification (RPA), loop-mediated amplification (LAMP), or nucleic acid sequence based amplification (NASBA), absorbance (e.g. aggregation of gold nanoparticles.

### Advantages of the Invention

The methods described herein provide several advantages over existing technologies such as the Micromagnetic Aptamer PCR (MAP) technology as described in Csordas et al. (Detection of Proteins in Serum by Micromagnetic Aptamer PCR (MAP) Technology. Agnew. Chem. Int. Ed. 2010 Vol 49 pages 355-358).

In the methods described herein, the aptamers are eluted from the target protein to proceed to amplification for detection. In contrast, MAP elutes the entire complex with beads in the PCR reaction which is a problem because: (i) MAP gets multiple aptamers per bead, which would make single molecule counting impossible; and (ii) the extra protein and bead surface hurt the efficiency of subsequent PCR reaction, and thereby limit the speed and sensitivity of detection.

Further, MAP requires continuous microfluidic washing whereas the methods described herein can avoid microfluidic washing, using a simple batch washing approach and still achieve high detection sensitivity with the antibody-aptamer sandwich.

Additionally, the ultimate signal from MAP is highly dependent on the elution volume (about 300 μL), and therefore is a major source of test-to-test variability. The methods described herein are not dependent on volume of the original sample. This is important because it is difficult to elute magnetic beads from a device with a fixed volume as it can vary depending on the flow rate, bubble formation, bead aggregation, magnetic adhesion to the channel surface, protein-based sticking to the surface, and fluidic losses. MAP mixes all the components at once at the start of the assay, including the capture antibody, target, and the aptamers. The methods described herein describe a multi-step immunoassay with multiple washes to decrease background binding. The instant methods do not require the use of magnetic beads whereas the MAP process requires them for the magnetic capture stage.

The quantity of beads has to be precisely controlled for MAP. Variations in the quantity of beads used in MAP results in variable capture, variable elution, and variable PCR efficiency, all leading to poor quantification.

MAP cannot use too many magnetic beads because it will block the chip and affect the washing efficiency. Continuous washing without loss of target protein requires binders with very slow off-rates. The methods described herein use batch washing, which does not have requirement on the kinetic properties of the binders allowing quantification of targets for which slow-off rate binders don't exist.

All performance metrics of digital measurement, including sensitivity, precision and dynamic range, improve with the total number of digital reactions. In digital ELISA, digitization of the immunocomplexes is subsequently achieved by loading the beads into arrays made from glass that contained 50,000 femtoliter wells (approximately 25,000-30,000 beads will be loaded into the wells). The methods described herein increase sensitivity by partitioning the detection objects (for example, detection aptamers) into up to 100 million separate droplet reactors, which is a 4,000 fold improvement over digital ELISA.

The number of beads loaded into the microwells in digital ELISA limits the assay dynamic range. In contrast, the methods described herein significantly improve the dynamic range of the assay because the methods described herein provide 4,000 times the number of partitions compared with digital ELISA.

In the instant methods, the partitioning of detection aptamers does not rely on beads. The methods described herein can partition the detection aptamers into reactions with different volumes; compartments of different volumes decouple the link between the total volume of all compartments and the size and number of smallest compartments. The smallest compartments enable quantification of high concentrations, while the compartments of large volumes enable high sensitivity by efficiently increasing the total volume. Using this multi-volume approach, the total number of compartments required for “digital” (single molecule) measurements can be minimized while maintaining high dynamic range and high resolution. This approach is also well suited for point-of-care purpose because it allows for simpler instrument design by minimizing the number of compartments for digital measurement and facilitates the development of new high-performance diagnostic tools for resource-limited applications.

In digital ELISA, large numbers of beads (typically 200,000-500,000) are used to capture the proteins in a sample to ensure that most beads capture only a single analyte molecule. This approach uses excessive capture reagents and therefore increases the background signal and limits the assay sensitivity due to three main sources: (i) nonspecific interactions of labeling reagents with the beads in the absence of target protein; (ii) interactions of molecules in the matrix (e.g., serum or plasma) that are endogenous and bind to beads, get labeled with enzymes, and yield false-positive signals; and (iii) interactions of molecules in the matrix (e.g., serum or plasma) that are immunologically derived, interact specifically with the capture or detection antibodies and give rise to false-positive signals, commonly known as heterophilic antibodies.

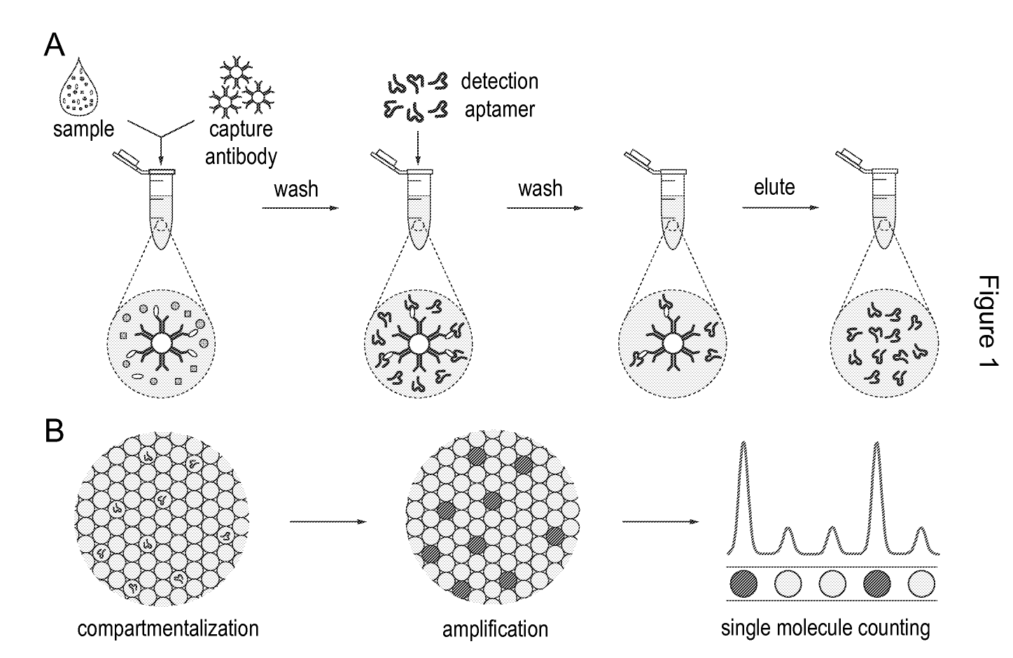

Using the instant methods, the front end (FIG. 1a) of the assay can be directly adapted from other conventional ELISA platforms except the adoption of aptamers as the detection reagent instead of antibodies. The background signal is significantly reduced using the methods described herein because no excessive amount of beads is needed, leading to improvement in limit of detection.

There are three steps of digital ELISA: incubation of capture beads with sample, incubation of captured protein with detection antibody, and incubation of detection-labeled protein with enzyme conjugate. The efficiency of each step can change significantly for different target analytes. A process of optimizing the concentrations of detection antibody and enzyme for fixed incubation times to ensure that there will only be one detection antibody on each bead (each compartment) and bring backgrounds down to the Poisson noise floor needs to be carried out every time a new assay is developed. Using the methods described herein, the eluted aptamers are monomeric in solution so they can be easily diluted and partitioned into millions of separate droplet reactors and quantified using any digital nucleic acid measurement methods without any specific optimization. Furthermore, the front end (FIG. 1a) of the assay is completely decoupled from and the back end (FIG. 1b) and we can adopt the optimized conditions from conventional ELISA experiments except for using detection aptamers instead of detection antibodies.

In digital ELISA, it is advantageous that the majority of the beads remain monomeric so that the beads can fit into microwells that are sized for single beads. Unfortunately, depending on coating conditions and the solubility properties of the coating antibody, beads may exhibit varying amounts of aggregation during antibody coupling. The coupling reaction conditions have to be optimized on the basis of maximizing antibody coupling efficiency while maintaining the bead in a monomeric state. This is not an issue for the methods described herein since the detection aptamers can be easily eluted from the solid supports, and then partitioned into millions of picoliter droplet reactors.

Biotinylation of detector antibodies and the preparation of streptavidin-enzyme conjugate for digital ELISA require extreme care. The signals and backgrounds in digital ELISA are dependent on the number of biotins incorporated; this number can vary widely with different detection antibodies. Commercial sources of streptavidin-enzyme conjugates are often aggregated, which has a dramatic impact on single molecule assays. Instead of high numbers of wells containing single enzymes, an aggregated streptavidin-enzyme conjugate will give rise to array images containing fewer, brighter wells, and can severely impact the detection efficiency of the assay. In the methods described herein, when an antibody-analyte-aptamer sandwich complex is formed on the solid support, the aptamer corresponds to a one-to-one representation of the target analyte. Due to this one aptamer per protein relationship, we can know exactly how many copies of the target analyte were in the initial sample through single aptamer (nucleic acid) counting method such as digital PCR.

Use of aptamers as the detection affinity reagents instead of antibodies can eliminate the specific interactions between the detection reagents and the molecules in the matrix (e.g., serum or plasma) that are immunologically derived, providing a solution to the commonly known heterophilic antibody problem.

The various methods and techniques described above provide a number of ways to carry out the application. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

### EXAMPLES

The following examples are not intended to limit the scope of the claims to the invention, but are rather intended to be exemplary of certain embodiments. Any variations in the exemplified methods which occur to the skilled artisan are intended to fall within the scope of the present invention.

### Example 1

### Aptamer-Based Ultrasensitive Assay for Tumor Necrosis Factor Alpha (TNFα)

The ELISA wells (Thermo Scientific, Catalog #: 15031) were functionalized with a TNFα antibody (eBioscience, Catalog #: 14-7349-85) at 5 μg/ml in PBS overnight at 4° C. Right before use, the ELISA wells were blocked with 300 μL AptaBuffer (PBS+0.05% Tween-20+2.5 mM MgCl₂+1 mM CaCl₂+1% BSA+0.5 mg/mL Dextran Sulfate+0.1 mg/mL Salmon Sperm DNA) with gentle shaking at ˜500 RPM for at least 30 minutes.

Next, test samples (50 μL) containing the protein of interest (TNFα, Shenandoah Biotechnology, Catalog #: 100-111) were added to the washed ELISA wells and incubated for 1 hour with gentle shaking After the target-capture incubation, the ELISA wells were washed once with PBSMCT (PBS+0.05% Tween-20+2.5 mM MgCl₂+1 mM CaCl₂) and 5 nM of the detection TNFα aptamer was added to the wells and incubated for 30 minutes with gentle shaking. The ELISA plate was then washed 4 times with 300 μl of PBS-MCT after the incubation with the detection aptamer. In an embodiment, the sequence of TNFα aptamer is 5′-ATCCAGAGTGACGCAGCATGCTTAAGGGGGGGGCGGGTTAAGGGAGTGGGGAG GGAGCTGGTGTGGACACGGTGGCTTAGT-3′.

For the elution step, 100 μL of PCR grade water was added to each ELISA well. After heating the plate at 95° C. for 10 minutes, we transfer the 100 μL of eluent containing the detection aptamers into a new tube. The eluents are diluted 10 fold before they are measured by the QuantStudio® 3D Digital PCR System from Life Technologies.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

It is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

1. A method for detecting target molecules or particles in a sample comprising:
(i) exposing the sample to a plurality of capture objects that each include a binding surface having affinity for the target molecules or particles, so as to form a complex between the capture objects and the target molecules or particles;
(ii) removing the capture objects not complexed with the target molecules or particles;
(iii) exposing the complex of capture object and target molecules or particles to a plurality of detection objects so as to form a complex between the capture objects, target molecules or particles and the detection objects;
(iv) removing the detection objects not complexed with the capture objects and the target molecules or particles;
(v) eluting the detection objects complexed with the capture objects and the target molecules or particles;
(vi) partitioning the detection objects into compartments; and
(vii) detecting the presence or absence of the detection objects in each compartment,
so as to detect the target molecules or particles in the sample.

2. The method of claim 1, wherein the sample is a fluid sample.

3. The method of claim 2, wherein the fluid sample is any of blood, plasma, sweat, serum or urine.

4. The method of claim 1, wherein the target is a protein, nucleic acid, small molecules, amino acids, carbohydrates, lipids, aminoglycosides, antibiotics, peptides, proteins, post-translational modification, nucleic acids or combinations thereof.

5. The method of claim 4, wherein the protein is a monomer or a multimer.

6. The method of claim 1, wherein the plurality of capture objects is any one or more of an antibody, an aptamer, a polypeptide, receptor, ligand, small molecule, or any other affinity reagents for the target molecules or a combination thereof.

7. The method of claim 1, wherein the plurality of detection objects is any one or more of an aptamer, an aptamer conjugated to an affinity agent, nucleic acid conjugated to affinity agent or a combination thereof,
wherein the affinity agent is any one or more of an antibody, a polypeptide, receptor, ligand, small molecule or a combination thereof; and
wherein the aptamer or the nucleic acid serve as the signal molecule for digital detection.

8. The method of claim 1, wherein the plurality of capture objects are antibodies and the plurality of detection objects are aptamers.

9. The method of claim 1, wherein the plurality of capture objects are aptamers and the plurality of detection objects are aptamers.

10. The method of claim 1, wherein the plurality of capture objects is bound to a plurality of solid support.

11. The method of claim 10, wherein the plurality of solid support are beads, nanoparticles, nanotubes (e.g., carbon nanotubes), microtiter plates, microfluidic channels, electrodes, vesicles, cells, film (e.g. nitrocellulose), tubing, or combinations thereof.

12. The method of claim 8, wherein aptamer is detected by digital detection methods.

13. The method of claim 12, wherein the digital detection methods are any one or more of digital polymerase chain reaction (PCR), digital rolling circle amplification (RCA), digital loop-mediated amplification (LAMP), Recombinase Polymerase Amplification (RPA) or digital nucleic acid sequence based amplification (NASBA).

14. The method of claim 8, wherein the antibody and the aptamer bind the same epitope.

15. The method of claim 8, wherein the antibody and the aptamer bind a different epitope.

16. The method of claim 1, wherein the eluted detection objects are partitioned into compartments such that each compartment consists of zero or one detection object.

17. The method of claim 16, wherein the compartment with one detection object is indicative of the presence of one target molecule.

18. The method of claim 1, wherein an absolute concentration of the target molecule in the sample is the total number compartments with one aptamer divided by the volume of the sample.

19. The method of claim 1, wherein concentration of target molecules or particles in the sample is less than about 50×10⁻¹⁵M, or less than about 40×10⁻¹⁵M, or less than about 30×10⁻¹⁵M, or less than about 20×10⁻¹⁵M, or less than about 10×10⁻¹⁵M, or less than about 5×10⁻¹⁵M, or less than about 1×10⁻¹⁵M.

20. The method of claim 1, wherein the concentration of target molecules or particles in the fluid sample is determined at least in part by comparison of a measure parameter to a calibration standard.

21. The method of claim 1, wherein at least about 10%, or at least about 20%, or at least about 30%, or at least about 40%, or at least about 50%, or at least about 60%, or at least about 70%, or at least about 80%, or at least about 90%, or at least about 95%, or at least about 99% or at least about 100% of the target molecules or particles bind to the capture objects so as to form a complex between the capture objects and the target molecules or particles.

22. The method of claim 1, wherein at least about 10%, or at least about 20%, or at least about 30%, or at least about 40%, or at least about 50%, or at least about 60%, or at least about 70%, or at least about 80%, or at least about 90%, or at least about 95%, or at least about 99% or at least about 100% of the complex between the capture objects and the target molecules or particles bind to the detection objects so as to form a complex between the capture objects, target molecules or particles and the detection objects.

23. A method for detecting target molecule in a sample comprising:
(i) exposing the sample to a solid support comprising a capture antibody specific for the target molecule so as to form a target-antibody complex;
(ii) removing the unbound sample and antibody;
(iii) exposing the target-antibody complex to a detection aptamer specific for the target;
(iv) removing the unbound aptamer;
(v) eluting the aptamer bound to the target-antibody complex;
(vi) partitioning the aptamers into compartments; and
(vii) detecting the presence or absence of an aptamer in each compartment,
so as to detect the target molecules in the sample.

24-40. (canceled)

41. A system for detecting target molecules or particles in a sample comprising:
(i) an array of reaction vessels wherein at least one of the reaction vessels contain no sample and at least one of the vessels contains a control sample that does not contain a target molecule or particle;
(ii) a plurality of capture objects that each include a binding surface having affinity for the target molecules or particles, so as to form a complex between the capture objects and the target molecules or particles;
(iii) plurality of detection objects so as to form a complex between the capture objects, target molecules or particles and the detection objects; and
(iv) an agent to elute the detection objects complexed with the capture objects and the target molecules or particles.

42-63. (canceled)

64. The method of claim 9, wherein aptamer is detected by digital detection methods.
